(19) United States
(12) Patent Application Publication
(10) Pub. No.: US 2012/0064161 A1
(43) Pub. Date: Mar. 15, 2012

(54) MODIFIED RELEASE NIACIN PHARMACEUTICAL FORMULATIONS

(75) Inventors: Rajesh Vooturi, Hyderabad (IN); Dhananjay Singare, Jalna (IN); Shantanu Yeshwant Damle, Thane (West) Thane (IN); Pradeep Jairao Karatgi, Hyderabad (IN); Sesha Sai Marella, Guntur (IN); Harshal Prabhakar Bhagwatwar, Hyderabad (IN); Ish Kumar Khanna, Alpharetta, GA (US); Raviraj Sukumar Pillai, Hyderabad (IN); Sivaram Pillarisetti, Norcross, GA (US)

(73) Assignees: DR. REDDY'S LABORATORIES, INC., Bridgewater, NJ (US); DR. REDDY'S LABORATORIES LTD., Hyderabad (IN)

(21) Appl. No.: 13/299,591

(22) Filed: Nov. 18, 2011

Related U.S. Application Data
(63) Continuation of application No. PCT/US2009/066333, filed on Dec. 2, 2009.

(30) Foreign Application Priority Data
May 18, 2009 (IN) 1144/CHE/2009

(51) Int. Cl. A61K 9/28; A61P 9/12; A61K 9/32; A61K 31/455; A61K 9/36

(52) U.S. Cl. 424/480; 514/356; 424/474; 424/482

(57) ABSTRACT

Pharmaceutical formulations comprising niacin in a matrix comprising a hydrophobic polymer that modifies release of niacin.

### INTRODUCTION

Aspects of the present invention relate to pharmaceutical formulations comprising niacin in modified, including extended, delayed, and delayed-extended release forms for oral administration. Methods of using the formulations of the invention to modulate niacin-induced flushing and hepatotoxicity are also included.

Dyslipidemia is elevation of plasma cholesterol and/or triglycerides (TG) or a reduced amount of high density lipoprotein (HDL) that contributes to the development of atherosclerosis. Causes may be primary (genetic) or secondary. Diagnosis is by measuring plasma levels of total cholesterol, TG, and individual lipoproteins. Treatment involves dietary changes, exercise, and lipid-lowering drugs. Diabetes is an especially significant secondary cause because patients tend to have an atherogenic combination of high TG, high small, dense low density lipoprotein (LDL) fractions, and low HDL (diabetic dyslipidemia, hypertriglyceridemic hyperapo B). Patients with type 2 diabetes are especially at risk. The combination may be a consequence of obesity and/or poor control of diabetes, which may increase circulating free fatty acids (FFA), leading to increased hepatic very low density lipoprotein (VLDL) production. TG-rich VLDL then transfers TG and cholesterol to LDL and HDL, promoting formation of TG-rich, small, dense LDL and clearance of TG-rich HDL. Diabetic dyslipidemia is often exacerbated by the increased caloric intake and physical inactivity that characterize the lifestyles of some patients with type 2 diabetes. Women with diabetes may be at special risk for cardiac disease from this form.

Niacin (nicotinic acid, or 3-pyridinecarboxylic acid) is a white, crystalline powder, very soluble in water, having structural Formula I.

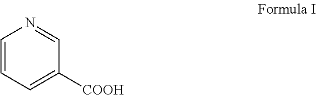

Niacin, when taken in large doses, has been shown to reduce levels of total cholesterol (TC), LDL and TG. It has also been shown to increase HDL levels in circulation and reduce cardiovascular risk in patients with documented cardiovascular disease. Multiple mechanisms have been proposed for the lipid modulating effects of niacin. It blocks or inhibits lipolysis in adipose tissue thus reducing free fatty acids in plasma. Niacin inhibits uptake of apolipoprotein A1 (apoA1) by the liver without affecting the clearance of cholesterol associated with HDL.

Commercially, niacin is available in immediate release (IR) formulations (such as NIACOR™ tablets from Upsher-Smith Laboratories, Inc.), each NIACOR™ Tablet, for oral administration, contains 500 mg of nicotinic acid. In addition, each tablet contains the following inactive ingredients: croscarmellose sodium, hydrogenated vegetable oil, magnesium stearate and microcrystalline cellulose.

Intermediate release formulations (such as NIASPAN™ tablets from Kos Pharmaceuticals Inc.), and sustained release (SR) formulations are commercially available. Niacin therapy is generally initiated at lower doses of about 375 or 500 mg and titrated to a maximum daily dose of 3 g. Even though IR niacin provides significant therapeutic efficacy in lowering TG and LDL and increasing HDL cholesterol, its widespread use is limited by the high incidence of cutaneous flushing that is not acceptable to patients. Sustained release formulations that were developed to reduce cutaneous flushing resulted in the generation of hepatotoxicity. Such formulations are available as dietary supplements, but are not approved by regulatory authorities world-wide for therapeutic uses. Formulations with release profile of niacin that is intermediate between IR and sustained release formulations were developed by Kos Pharmaceuticals Inc. These formulations, commercially available as NIASPAN™, provide equivalent or better therapeutic efficacy when compared with IR niacin and reduced flushing when compared with the IR formulations and low hepatotoxicity compared to sustained release formulations. A comparative clinical study was conducted by Kos Pharmaceuticals Inc., wherein doses of 1500 mg of niacin IR and its intermediate release formulations (NIASPAN tablets) were administered for a period of about 16 weeks, demonstrating equivalent or improved performance of the intermediate release formulations when compared with IR niacin.

Each NIASPAN tablet, for oral administration, contains 500, 750, or 1000 mg of nicotinic acid. In addition, NIASPAN tablets also contain the inactive ingredients hypromellose (hydroxypropyl methylcellulose, or HPMC), povidone, stearic acid, and polyethylene glycol, and the coloring agents FD&C yellow #6/sunset yellow FCF Aluminum Lake, synthetic red and yellow iron oxides, and titanium dioxide, where the hypromellose is a release rate controlling agent of the hydrophilic polymer type.

NIASPAN is indicated as an adjunct to diet for the reduction of elevated TC, LDL-C, apolipoprotein B and TG levels, and to increase HDL in patients with primary hypercholesterolemia and mixed dyslipidemia. NIASPAN is also indicated to reduce the risk of recurrent nonfatal myocardial infarction and to slow the progression or promote the regression of atherosclerotic disease. NIASPAN is to be taken at bedtime, after a low-fat snack, and doses are individualized according to patient response.

High doses of niacin have been shown to elevate fasting blood sugar levels, thereby worsening type 2 diabetes. Accordingly, niacin is contraindicated for persons with type 2 diabetes. The mechanism behind niacin-induced insulin resistance and diabetes is presently unknown. U.S. Pat. Nos. 5,126,145, 5,268,181, 6,080,428, 6,129,930, 6,406,715, 6,469,035, 6,676,967, 6,746,691, 6,818,229, and 7,011,848 disclose sustained and intermediate release formulations of nicotinic acid. U.S. Pat. No. 5,981,555, U.S. Patent Application Publication Nos. 2004/0053975 and 2005/0148556, and International Application Publication Nos. WO 2004/103370, WO 2006/017354, WO 2007/041499, WO 2004/111047, and WO 2009/005803 describe methods of reducing niacin-induced flushing.

From the discovery of niacin as an agent to treat dyslipidemia to the present date, continuous efforts have been made to improve the performance of the drug in oral dosage forms. As a result, sustained and intermediate release dosage forms have evolved, in addition to the IR formulation. It is now well reported that the pharmacokinetics and metabolic profile of niacin is influenced by the rate of niacin administration, this in turn governs the pharmacokinetics of the metabolites including nicotinuric acid (NUA), nicotinamide (NAM), and nicotinamide N-oxide. Cₘₐₓ and AUC₀₋ₜ for niacin and nicotinuric acid increases with an increase in dosing rates: similarly, it also results in significant increase in the urine recovery of niacin and nicotinuric acid, along with a significant decrease in N-methyl-2-pyridone-5-carboxamide (2PY) and N-methylnicotinamide (MNA).

Most of the literature indicates a pressing need for improvement in the performance of niacin in terms of its safety and efficacy. The introduction of NIASPAN into the market addressed some of the concerns with prior niacin therapy, like providing an intermediate release profile to control flushing and minimize hepatotoxicity. However in placebo-controlled clinical trials for NIASPAN, flushing episodes, i.e., warmth, redness, itching and/or tingling, were the most common treatment emergent adverse events, reported by as many as 88% of patients, with 47% of the patients dropping out of the study due to flushing (source: summary basis of approval for NIASPAN).

A need exists for niacin-containing formulations that reduce the incidence of flushing, while providing benefits equivalent to commercially available formulations. A need exists for niacin-containing formulations that provide statistically significant exposures of niacin at the same time providing statistically significant lower levels of plasma and urinary metabolites that are responsible for hepatotoxicity when compared with a commercially available intermediate release formulation (NIASPAN).

### SUMMARY

Aspects of the present invention relate to modified release formulations of niacin for oral administration.

In embodiments, the invention provides modified release formulations of niacin comprising:

(a) a niacin-containing core comprising a therapeutically effective amount of niacin or its salt or a prodrug, a pharmaceutically acceptable release controlling agent, and another pharmaceutically acceptable excipient;

(b) optionally, a barrier coating layered over the niacin-containing core; and

(c) optionally, an enteric coating applied directly onto either the core, if (b) is not present, or onto the barrier coating.

In embodiments, the invention provides modified release formulations of niacin, wherein a niacin-containing core includes a hydrophobic polymer as a release controlling agent.

In embodiments, the invention provides modified release formulations of niacin, wherein a niacin-containing core includes a hydrophobic polymer such as a copolymer of alkyl acrylate and alkyl methacrylate (e.g., a EUDRAGIT® product), a cellulose acetate, or zein, as a release controlling agent.

In embodiments, the invention includes modified release formulations of niacin, comprising:

(a) a niacin-containing core comprising a therapeutically effective amount of niacin, its salt or a prodrug, and a pharmaceutically acceptable excipient;

(b) optionally, a barrier coating layered onto the core; and

(c) optionally, an enteric coating applied directly onto the core, if (b) is not present, or onto the barrier coating.

In embodiments, the invention provides modified release formulations of niacin, wherein the formulations provide statistically significantly higher niacin exposure in plasma or blood, as compared to exposure obtained after oral administration of a similar amount of niacin from the commercially available NIASPAN intermediate release niacin product.

In embodiments, the invention provides modified release formulations of niacin, wherein the formulations provide statistically significantly higher or equivalent exposure in plasma, as compared to exposure obtained after oral administration of a higher amount of niacin from the commercially available NIASPAN intermediate release niacin product.

In embodiments, the invention provides modified release formulations of niacin, wherein the formulations provide statistically significant increases in niacin levels in plasma or blood (i.e., Cₘₐₓ and/or AUC), as compared to those obtained after oral administration of a similar amount of niacin from the commercially available NIASPAN intermediate release niacin product.

In embodiments, the invention provides modified release formulations comprising niacin in a matrix comprising a hydrophobic polymer, wherein the formulations, following administration to a healthy human, provide at least one of the pharmacokinetic parameters Cₘₐₓ and AUC greater than a corresponding value obtained from administering a pharmaceutical formulation containing 50 percent more niacin in a matrix comprising a hydrophilic polymer.

In embodiments, the invention provides modified release formulations of niacin, providing statistically significant higher or equivalent plasma niacin levels (i.e, Cₘₐₓ and/or AUC), as compared to those obtained after oral administration of a higher amount of niacin from the commercially available NIASPAN intermediate release niacin product.

In embodiments, the invention further provides formulations comprising modified release niacin, the formulations providing at least a two-fold increase in Cₘₐₓ and AUC, as compared to those obtained after oral administration of an equivalent amount of niacin from the commercially available NIASPAN intermediate release niacin product.

In embodiments, the invention further provides modified release formulations of niacin, wherein the formulations provides higher statistically significant exposure in plasma, as compared to those obtained after oral administration of an equivalent amount of niacin from the commercially available NIASPAN intermediate release niacin product.

The presence of a barrier coating, applied between a niacin-containing core and an enteric coating, is an embodiment of the invention that can provide significantly higher systemic exposure of the niacin, upon administration to a mammal in need of administration of niacin.

In certain embodiments, the modified release formulations release their contained niacin at a slower rate into an aqueous fluid than is obtained with an immediate release formulation, but at a faster rate than is obtained with intermediate release and sustained release formulations known in the art, when tested under similar dissolution conditions. In other embodiments, modified release formulations provide an in vitro release of their contained niacin at rates substantially equivalent to prior art formulations, when tested under similar dissolution conditions.

In certain embodiments, the modified release formulations of niacin release niacin at a slower rate, and/or with commencement of release delayed for a time, such as during about the first 60 minutes after oral dosing, which allows the simultaneous administration of anti-flushing agents to help control the flushing caused by niacin. For example, the present compositions allow for the simultaneous administration of flush-inhibiting agents such as non-steroidal anti-inflammatory agents (NSAIDs), cyclooxygenase-2 inhibitors, PGD2-antagonists, or other compounds with similar activity, together with the modified release niacin formulation. The provision of slower and/or delayed release of the niacin with a co-administration or simultaneous immediate release flush-inhibiting agent allows for levels of the flush-inhibiting agent to build up in the body before peak concentrations of niacin are obtained. Subsequent extended release provided by the release controlling agents in the niacin containing core provides sustained high levels of the niacin in the body.

In embodiments, the invention provides pharmaceutical formulations comprising niacin in a matrix that modifies release of niacin by incorporating a hydrophobic material, the formulations providing, following administration to a healthy human, at least one of the pharmacokinetic parameters Cₘₐₓ and AUC_{0-∞} greater than a corresponding value obtained from administering a pharmaceutical formulation containing a similar or greater amount of niacin in a matrix that modifies release of niacin by incorporating a hydrophilic polymer.

In embodiments, the invention provides pharmaceutical formulations comprising niacin in a matrix that modifies release of niacin by incorporating a hydrophobic material, and a first polymer coating disposed over a tablet formed using the matrix, the formulations providing, following administration to a healthy human, at least one of the pharmacokinetic parameters Cₘₐₓ and AUC_{0-∞} greater than a corresponding value obtained from administering a pharmaceutical formulation containing a similar or greater amount of niacin in a matrix that modifies release of niacin by incorporating a hydrophilic polymer.

In certain embodiments, the invention includes processes for manufacturing formulations of the invention, as well as methods of using the formulations for the treatment of a variety of disease conditions.

### BRIEF DESCRIPTION OF THE DRAWINGS

FIG. 1 is a graph showing the in vitro dissolution profiles of products from Examples 1, 2, 3, 4 and 5.

FIG. 2 is a graph showing an in vitro dissolution profile of the product from Example 6.

FIG. 3 is a graph showing the in vitro dissolution profiles of the products from Examples 7 of the present application, NIACOR 500 mg tablets, and NIASPAN 500 mg tablets.

FIG. 4 is a graph showing mean plasma niacin concentrations obtained on day 1 in a pharmacokinetic study with formulations of Example 10 (A) and NIASPAN tablets (B).

FIG. 5 is a graph showing mean plasma niacin concentrations obtained on day 3 in a pharmacokinetic study with formulations of Example 10 (A) and NIASPAN tablets (B).

FIG. 6 is a graph showing mean plasma nicotinuric acid (NUA) concentrations obtained on day 1 in a pharmacokinetic study with formulations of Example 10 (A) and NIASPAN tablets (B).

FIG. 7 is a graph showing mean plasma NUA concentrations obtained on day 3 a pharmacokinetic study with formulations of Example 10 (A) and NIASPAN tablets (B).

FIG. 8 is a graph showing mean plasma nicotinamide (NAM) concentrations obtained on day 1 in a pharmacokinetic study with formulations of Example 10 (A) and NIASPAN tablets (B).

FIG. 9 is a graph showing mean plasma NAM concentrations obtained on day 3 in a pharmacokinetic study with formulations of Example 10 (A) and NIASPAN tablets (B).

### DETAILED DESCRIPTION

Aspects of the present invention relate to modified release formulations of niacin for oral administration.

The modified release formulations of the present invention with their unique in vitro and in vivo release profiles can provide effective niacin therapy with the benefits of reduced flushing, when compared with prior niacin formulations.

The term “niacin” is intended to include niacin free acid and any of its pharmaceutically acceptable salts, solvates, hydrates, polymorphs or mixtures thereof, or a prodrug for niacin in its free acid or base form or its pharmaceutically acceptable salts, solvates, hydrates, polymorphs or mixtures thereof. As used herein, the term “prodrug” encompasses compounds other than niacin, which the body metabolizes into niacin, thus producing the same effect as described herein. For example, the liver can synthesize niacin from the essential amino acid tryptophan. Other compounds specifically include, but are not limited to, nicotinamide, nicotinyl alcohol tartrate, d-glucitol hexanicotinate, aluminum nicotinate, niceritrol, and d, 1-alpha-tocopheryl nicotinate.

“Formulation” in the context of present invention refers to a unit dose pharmaceutical formulation comprising niacin which is in a solid dosage form, examples of solid dosage forms including, but not limited to, monolithic tablets, bilayered or multi layered tablets, capsules, tablets in capsules, and pellets, granules, or particles filled into capsules or compressed into tablets.

“Modified release” is intended to mean formulations which provide slow, delayed, extended or delayed-extended release of niacin, without limitation. Any mechanism of providing delayed, extended or delayed-extended release is included within the scope of the invention as long as the basic principles of the invention are met, including combinations of two or more of the above release types, but excluding the intermediate release profiles of a NIASPAN product.

Formulations comprising niacin as described herein can provide one or more of the following advantages over prior formulations:

a) Significant reduction in niacin-induced flushing relative to immediate release niacin formulations.

b) Equivalent or reduced niacin-induced hepatotoxicity, when compared with the known sustained release formulations, or comparable with that of NIASPAN tablets of similar strength.

c) Statistically significant exposures of niacin as compared NIASPAN tablets of similar strength.

d) Improved dyslipidemia or anti-atherosclerotic profiles, as compared to other formulations.

Formulations comprising niacin as described herein can provide one or more of the following advantages over immediate release niacin formulations:

a) Significant reduction in niacin-induced flushing at comparable doses.

b) Sustained drug levels over an extended period, and a potential for once- or twice-daily dosing.

c) Improved or comparable efficacy for dyslipidemic patients at comparable doses.

Formulations comprising niacin as described herein can provide one or more of the following advantages over known sustained release niacin formulations:

a) Significant reduction in niacin-induced hepatotoxicity at comparable doses.

b) Improved efficacy for dyslipidemic patients at comparable doses.

Formulations comprising niacin as described herein can provide one or more of the following advantages over intermediate release formulations such as NIASPAN:

a) Improved plasma niacin concentrations (AUC and/or Cₘₐₓ) after administering comparable doses.

b) Comparable or reduced flushing at comparable plasma niacin concentrations (Cₘₐₓ and/or AUC).

c) Comparable or enhanced efficacy at similar doses.

Embodiments of the invention include modified release pharmaceutical formulations of niacin, comprising:

(a) a niacin-containing core comprising a therapeutically effective amount of niacin or a prodrug, a pharmaceutically acceptable release controlling agent, and one or more other pharmaceutically acceptable excipients;

(b) optionally, a barrier coating applied onto the core; and

(c) optionally, an enteric coating applied directly onto the core, if (b) is not present, or onto the barrier coating.

In embodiments, the invention provides modified release pharmaceutical formulations of niacin, comprising:

(a) a niacin-containing core comprising a therapeutically effective amount of niacin, its salt or a prodrug, and one or more other pharmaceutically acceptable excipients;

(b) optionally, a barrier coating applied onto the core; and

(c) optionally, an enteric coating applied directly onto the core, if (b) is not present, or onto the barrier coating.

In embodiments of the invention, a niacin-containing core is provided comprising niacin, its salt or a prodrug, and pharmaceutically acceptable excipients. Where an immediate release core is desired, conventional pharmaceutical excipients for preparing a solid oral dosage forms will be used, such as diluents, binders, lubricants, and the like. Other such ingredients which are required for processing are also within the scope of the invention.

Where a controlled or extended release niacin-containing core is desired, a pharmaceutically acceptable release controlling agent will be added.

In embodiments, the invention provides modified release formulations of niacin, wherein a niacin-containing core comprises a hydrophobic polymer as a release-controlling agent.

Some hydrophobic materials which can be used are not polymers, including, but not limited to, fatty substances such as stearic acid and its metal salts, hydrogenated castor oil, glyceryl monostearate, and glyceryl behenate, minerals such as talc, etc. Other hydrophobic materials can also be used, meaning the invention is not limited to the use of only hydrophobic polymers.

Release-controlling polymers, in the context of the present invention, include hydrophobic polymers, delayed release (e.g., enteric) polymers, bioadhesive (or mucoadhesive) polymers, hydrophobic substances like waxes and fats, and combinations thereof. The content of release-controlling polymer in the formulations of the present invention may vary from about 1% to about 90% or from about 5% to about 80%, of the total weight of the formulation.

Useful hydrophobic polymers or combinations thereof used in various ratios include, but are not limited to: cellulose derivatives such as methylcelluloses, ethylcelluloses, cellulose acetates and their derivatives, cellulose acetate phthalate, hydroxypropyl methylcellulose phthalates, cellulose acylates, cellulose diacylates, cellulose triacylates, cellulose acetate, cellulose diacetate, cellulose triacetate, mono-, di- and tri-cellulose alkanylates, mono-, di-, and tri-cellulose arylates, and mono-, di- and tri-cellulose alkenylates; crosslinked vinylpyrrolidone polymers (crospovidone); polymethacrylic acid based polymers and copolymers such as are sold by Evonik Industries Ltd., Essen, Germany as EUDRAGIT™ (including Eudragit RL and RS, and NE-30D); zein; and aliphatic polyesters. This list is not intended to be exhaustive, as other classes of polymers, copolymers of these polymers, or their mixtures in various ratios and proportions are within the scope of this invention without limitation.

Hydrophobic substances such as waxes and fats may have melting points about 30° C. to about 200° C., or about 45° C. to about 90° C. Useful hydrophobic substances can include neutral or synthetic waxes, fatty alcohols (such as lauryl, myristyl, stearyl, cetyl or cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, and combinations comprising two or more of the foregoing materials. Suitable waxes include beeswax, paraffin wax, carnauba wax, etc., and also synthetic waxes such as, for example, microcrystalline waxes and other commercially available waxes, castor wax, wax-like substances, e.g., materials normally solid at room temperature and having a melting point of about 30° C. to about 100° C., and combinations comprising two or more of the foregoing.

In other embodiments, a niacin-containing core is an extended release core. In an embodiment, the release of niacin from the extended release niacin core is controlled using a hydrophobic material alone. In an embodiment, the hydrophobic material is a pH-independent polymeric material, such as, for example, a methacrylic acid polymer or a material such as an ethylcellulose, a wax such as carnauba wax, glyceryl monostearate, and the like. The niacin-containing core is further coated with an enteric coating. An enteric coating is provided to delay the release of the niacin from the immediate release or modified release core to allow the delivery of the niacin at a time point intermediate between that provided by immediate release formulations and extended release formulations. The provision of an enteric coating and excipients provide a formulation which generates pharmacokinetic profiles of niacin in the body upon administration to a mammal in need thereof, which are distinct from any pharmacokinetic profiles of known niacin formulations The exposure of niacin achieved in the body as measured by the area under the plasma concentration-time curves (AUC) obtained after administration of the formulations of the invention to a mammal, is significantly higher than that achieved upon administration of either the sustained release formulations or extended release formulations known in the art and commercially available. This is possibly due to unique characteristics of formulations of the invention.

Of course, any other release-controlling polymers, which demonstrate similar characteristics, are also acceptable in the working of this invention.

In another embodiment, the invention provides modified release formulations of niacin, wherein a niacin-containing core comprises a hydrophobic polymer such as a copolymer of an alkyl acrylate and alkyl methacrylate, e.g., a EUDRAGIT product, a cellulose acetate, or zein as a release controlling agent.

The niacin-containing core can be in the form of niacin-containing tablets of a variety of sizes and shapes, mini-tablets, non-pareil seed materials onto which the niacin is coated, or niacin pellets prepared by granulation or extrusion, together with pharmaceutically acceptable excipients, release controlling materials, and the like. The preparation of such drug-containing cores is within the scope of understanding of a person skilled in the art. The type and amounts of the release controlling materials will determine the duration of release of niacin that will be provided by the niacin-containing core, such for example an immediate release of niacin over a few minutes, or a release similar to that of NIASPAN, or any intermediate dissolution profile that is desired.

Thus according to yet another embodiment, there are provided low dose niacin formulations comprising modified release niacin as described herein, which formulations provide, following administration to a mammal, at least a 50% increase in Cₘₐₓ and/or AUC, when compared with a commercially available intermediate release product NIASPAN of similar strength.

In embodiments of the invention, the formulations result in a significant reduction in the dose of niacin required to provide a therapeutic effect. Modified release pharmaceutical formulations of the present invention exhibit a slow rate of drug release as compared to immediate release formulations (formulations having an in vitro release of more than about 75% of the contained niacin in less than about 2 hours, or in about 1 hour, when tested using a customary dissolution testing method in media such as pH 6.8 phosphate buffer, pH 7.5 phosphate buffer, etc.).

As used herein, a “therapeutically effective amount” is an amount that has a reasonable risk-to-benefit ratio for the treatment of certain diseases in the subjects in need thereof. A “therapeutically effective amount of niacin,” in the context of the present invention, includes about 250 mg, about 500 mg, about 750 mg, about 1000 mg, about 1500 mg, about 2000 mg, about 2500 mg, or about 3000 mg of niacin daily.

Niacin is extensively metabolized to produce different metabolites, the metabolism involving two pathways denoted pathway 1 and pathway 2.

Pathway 1 produces a nicotinuric acid (NUA) metabolite. Flushing is a significant adverse effect caused by this metabolite; the higher the plasma levels of NUA, the greater will be the degree of cutaneous flushing.

Pathway 2 produces nicotinamide (NAM), 6-hydroxy nicotinamide (6NH), nicotinamide —N-oxide (MNO), N-methyl nicotinamide (MNA), nicotinamide adenine dinucleotide (NAD) and 2-PY metabolites. Hepatotoxicity is the major adverse effect caused as a result of these metabolites; the higher the plasma levels of these metabolites, the greater will be the degree of hepatotoxicity.

Under in vivo conditions, pathway 2 is a high affinity, low capacity path and generates metabolites through phase I reactions. The pathway 1 metabolites are generated through a low affinity, high capacity conjugation path. The formulations of the invention can provide a plasma and urine metabolite profile which is distinct

In embodiments, the niacin from modified release formulations is released after a delay of about 10 minutes, about 20 minutes, about 30 minutes, about 1 hour, about 2 hours, about 4 hours, etc., after a dosage form enters into aqueous media.

In embodiments, the niacin-containing core is an immediate release core, with an enteric coating applied directly onto the core.

An enteric coating is a coating that prevents release of an active agent until the dosage form reaches a pH environment higher than that of the stomach. A delayed release dosage form comprises niacin and is coated with an enteric polymer. The enteric polymer should be non-toxic and is predominantly soluble in intestinal fluids, but substantially insoluble in the gastric juices. Examples of such delayed release (enteric) polymers include polyvinylacetate phthalates (PVAP), hydroxypropyl methylcellulose acetate succinates (HPMCAS), cellulose acetate phthalates (CAP), methacrylic acid copolymers, hydroxypropyl methylcellulose succinates, cellulose acetate succinates, cellulose acetate hexahydrophthalates, hydroxypropyl methylcellulose hexahydrophthalates, hydroxypropyl methylcellulose phthalates (HPMCP), cellulose propionate phthalates, cellulose acetate maleates, cellulose acetate trimellitates, cellulose acetate butyrates, cellulose acetate propionates, methacrylic acid/methacrylate polymers (acid number 300 to 330 and also known as EUDRAGIT L), which are anionic copolymers based on methacrylate and available as a powder (also known as methacrylic acid copolymer, type A NF, methacrylic acid-methyl methacrylate copolymer, ethyl methacrylate-methylmethacrylate-chlorotrimethylammonium ethyl methacrylate copolymer, and the like), and combinations comprising one or more of the foregoing enteric polymers. Other examples include natural resins, such as shellac, copal collophorium, and combinations comprising one or more of the foregoing polymers. Further examples of enteric polymers include synthetic resins bearing carboxyl groups. The methacrylic acid-ethyl acrylate 1:1 copolymer with an average molecular weight about 250,000 and sold as EUDRAGIT L 100-55 is suitable.

According to an aspect of the invention, significant pharmacokinetic advantages are provided by the formulations of the invention which comprise a barrier coating interposed between the niacin-containing core and the enteric coating. Without being bound by any particular theory, it is possible that the barrier coating prevents chemical interactions between the niacin from the core and the enteric coating material, which could result in significantly reduced release of the niacin from the formulation.

A barrier coating may be optionally applied to a core formulation to prevent interactions between the drug and enteric coating. It can also impart moisture protection to the core formulation.

Non-limiting examples of useful barrier coating materials include hydrophilic materials such as, but not limited to, sodium carboxymethylcelluloses, hydroxypropylcelluloses, hydroxyethylcelluloses, hydroxypropyl methylcelluloses, carboxymethylamide, potassium methacrylatedivinylbenzene co-polymers, polymethylmethacrylates, polyvinylpyrrolidones, polyvinylalcohols, methylcelluloses, carboxymethylcelluloses, polyoxyethylene glycols, xanthan gum, carbomers, POLYOX™ poly(ethylene oxide) polymers, hydrocolloids such as natural or synthetic gums, cellulose derivatives other than those listed above, carbohydrate-based substances such as acacia, gum tragacanth, locust bean gum, guar gum, agar, pectin, carrageen, soluble alginates, carboxypolymethylene, and the like, potassium methacrylate/divinylbenzene copolymers, polyhydroxyalkyl methacrylates, cross-linked polyvinylpyrrolidones, other substances such as arbinoglactan, pectin, amylopectin, gelatin, N-vinyl lactams, polysaccharides, and the like. Combinations of any two or more of these polymers, and other polymers having the required properties also are within the scope of the invention.

A bioadhesive polymer may be included in oral dosage forms to increase the contact time between the dosage form and the mucosa of a drug-absorbing section of the gastrointestinal tract. Non-limiting examples of bioadhesives include carbomers (various grades), sodium carboxymethylcelluloses, methylcelluloses, polycarbophils (e.g., NOVEON™ products), hydroxypropyl methylcelluloses, hydroxypropyl celluloses, sodium alginate, sodium hyaluronate, and combinations comprising any two or more of the foregoing.

Other inert materials, which can act as barriers to prevent interactions between the niacin and the enteric or functional polymer, are within the scope of the invention without limitation. The determination of the thickness of the barrier coating as well as the viscosity grade of a polymeric material, if used, are within the understanding of a person skilled in the art. Thus, when a polymeric material such as a HPMC is used, a suitable grade could include a low viscosity grade capable of acting as a barrier between the niacin and the enteric coating material without impacting the dissolution and release of the niacin upon contact with an aqueous medium. When a sugar is used as a barrier coating, the thickness of the coating will determine the degree of protection that such a coating will provide, as also will the type of sugar. Such and other aspects of selection of a barrier coating are within the scope of understanding of a person skilled in the art of preparation of solid oral dosage forms.

Other materials that can be used to prevent undesired interactions between the niacin-containing core and the enteric coating include acidic materials such as for example citric acid, ascorbic acid, tartaric acid, benzoic acid, and amino acids, such as, for example, aspartic acid, and glutamic acid. Other materials that can provide an acidic environment and prevent interaction between niacin and the enteric coating are also included within the scope of the invention without limitation. The acidic stabilizing materials can be blended with the ingredients before the niacin-containing core is compressed, or the materials can be layered onto a niacin-containing core to prevent interaction between the niacin-containing core and the enteric coating. The acidic materials can be used with niacin in weight ratios of acidic material to niacin about 0.01:1 to 0.5:1.

Any barrier material that is used and, whatever the mechanism by which the barrier coat acts to prevent the undesired interaction between the niacin-containing core and the enteric coat may be, the surprisingly high exposure levels provided by the inventive formulation will follow. Any such barrier coating is thus within the scope of the invention without limitation.

In some embodiments of the present invention, pharmaceutically acceptable excipients serving as pharmaceutically inert cores comprise: insoluble inert materials, such as glass particles/beads or silicon dioxide, calcium phosphate dihydrate, dicalcium phosphate, calcium sulfate dihydrate, microcrystalline cellulose (MCC) or cellulose derivatives; soluble cores such as acid cores like tartaric acid and spheres of sugars like sucrose, dextrose, lactose, anhydrous lactose, spray-dried lactose, lactose monohydrate, mannitol, starches, sorbitol, sucrose; insoluble inert polymeric materials such as spherical or nearly spherical core beads of polyvinyl chloride, polystyrene, or any other pharmaceutically acceptable insoluble synthetic polymeric material; and the like and mixtures thereof.

Modified release formulations comprising niacin and a release-controlling polymer may be prepared by any suitable techniques, including those described below. The active agent and a release-controlling polymer may, for example, be prepared by wet granulation techniques, dry granulation, direct compression, melt extrusion techniques, etc. The active agent in modified release formulations can include a plurality of substrates comprising the active ingredient, which substrates are coated with a sustained-release coating comprising a release-controlling polymer. The modified release formulations may thus be made in conjunction with a multiparticulate system, such as beads, ion-exchange resin beads, spheroids, microspheres, seeds, pellets, granules, and other multiparticulate systems in order to obtain a desired modified (delayed-extended) release of the active agent. The multiparticulate systems can be presented in a tablet or capsule or other suitable unit dosage form. In certain cases, more than one multiparticulate system can be used, each exhibiting different characteristics, such as pH dependence of release, time for release in various media (e.g., acidic, basic, simulated intestinal fluids), release in vivo, size, and formulation.

In some cases, a spheronizing agent, together with the active ingredient, can be spheronized to form spheroids. Microcrystalline cellulose and hydrous lactose impalpable are examples of such agents. Additionally (or alternatively), the spheroids can contain a water insoluble polymer, such as an acrylic polymer, an acrylic copolymer, such as a methacrylic acid-ethyl acrylate copolymer, or an ethyl cellulose. In this formulation, the release-modifying coating will generally include a water insoluble material such as a wax, either alone or in admixture with a fatty alcohol, or shellac or zein. Spheroids or beads, coated with an active ingredient can be prepared, for example, by dissolving or dispersing the active ingredient in a solvent and then spraying the solution onto a substrate, for example, sugar spheres NF-21, 18/20 mesh, using a Würster insert. Optionally, additional ingredients are also added prior to coating the beads in order to enhance the active ingredient binding to the substrates, and/or to color the resulting beads, etc. The resulting substrate-active material may optionally be over-coated with a barrier material, to separate the therapeutically active agent from the next coating of material, e.g., a release-controlling polymer.

The pharmaceutical formulations of the present invention can be prepared by various other methods and techniques as known to the skilled person, so as to achieve desired in vitro drug release profiles. Specific embodiments of processes include, but are not limited to, any of:

1. Direct compression, using appropriate punches and dies, the punches and dies being fitted to a suitable tableting press.

2. Injection or compression molding using suitable molds fitted to a compression unit.

3. Granulation followed by compression.

4. Extrusion in the form of a paste, into a mold or as an extrudate to be cut into desired lengths.

When particles are made by direct compression, the addition of lubricants may be helpful and sometimes this is important to promote powder flow and to prevent capping of the compressed particle (breaking off of a portion of the particle) when compression pressure is relieved. Typically, lubricants are added in amounts from 0.25% to 3% by weight. Additional excipients may be added to enhance powder flowability and reduce adherence.

Oral dosage forms may be prepared to include an effective amount of melt-extruded subunits in the form of multiparticulates within a capsule. For example, a plurality of the melt-extruded muliparticulates can be placed in a gelatin capsule in an amount sufficient to provide an effective release dose when ingested and contacted by gastric fluid. The subunits, e.g., in the form of multiparticulates, can be compressed into oral tablets using conventional tableting equipment using standard techniques.

The formulations may be in the form of minitablets or microtablets, enclosed inside a capsule, e.g., a gelatin capsule. For this, any gelatin capsule employed in the pharmaceutical formulation field can be used, such as the hard gelatin capsules known as CAPSUGEL™, available from Pfizer.

In embodiments, pharmaceutical formulations of the present invention can be prepared using a granulation process comprising:

a) dissolving or dispersing the active ingredient optionally with binder and/or solubilizer in a solvent;

b) granulating the pharmaceutically acceptable excipient blend with the solution comprising active;

c) drying and lubricating the granules; and

d) compressing the granules into tablets, or alternatively filling into capsules.

In other embodiments, pharmaceutical formulations of the present invention can be prepared using a direct compression process comprising:

a) mixing the active ingredient and a release-controlling polymer, optionally with other pharmaceutically acceptable excipients; and

b) compressing the blend of a) into tablets, or alternatively filling into capsules.

Alternatively, the formulations of the present invention can be prepared by dissolving the active ingredient in a suitable solvent, and coating the dissolved active, optionally with other excipients, onto the surface of inert core particles such as tartaric acid and the like as described above. Such drug-layered cores or pellets may further be granulated or coated with a release-controlling polymer to produce pharmaceutical formulations of the present invention.

The granules/beads or tablets or capsules may further be coated with a release-controlling polymer, optionally with other excipients. Such coating can be done using various known techniques such as dip coating, pan coating, fluidized bed coating, and the like.

The residual solvent content of the pharmaceutical formulations, as described herein, may be made low, such as less than about 5000 ppm by weight. The concentration of residual solvents can further be reduced to desired limits, as are acceptable by regulatory authorities, such as using drying steps.

In the context of the present invention, during the processing of the pharmaceutical formulations into finished dosage forms, one or more pharmaceutically acceptable excipients may optionally be used, including but not limited to: diluents such as microcrystalline cellulose (“MCC”), silicified MCC (e.g., PROSOLV™), microfine cellulose, lactose, starch, pregelatinized starches, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide, and the like; cores/beads such as insoluble inert materials like glass particles/beads or silicon dioxide, calcium phosphate dihydrate, dicalcium phosphate, calcium sulfate dihydrate, microcrystalline cellulose, cellulose derivatives; soluble cores such as sugar spheres of sugars like dextrose, lactose, mannitol, starches, sorbitol, or sucrose; insoluble inert polymeric materials such as spherical or nearly spherical core beads of polyvinyl chloride, polystyrene or any other pharmaceutically acceptable insoluble synthetic polymeric material, and the like or mixtures thereof; binders or adherents such as acacia, guar gum, alginic acid, dextrin, maltodextrin, methylcelluloses, ethylcelluloses, hydroxyethyl celluloses, hydroxypropyl celluloses (e.g., KLUCEL®), carboxymethyl cellulose sodium, povidones (various grades of KOLLIDON®, PLASDONE®), starches and the like; disintegrants such as carboxymethyl cellulose sodium (e.g., Ac-Di-Sol®, PRIMELLOSE®), crospovidones (e.g., KOLLIDON®, POLYPLASDONE®), povidone K-30, polacrilin potassium, starches, pregelatinized starches, sodium starch glycolate (e.g. EXPLOTAB®), and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like. Solvents that may be used in formulation processing include, for example, water, methanol, ethanol, isopropyl alcohol, acetone, methylene chloride, dichloromethane, and the like, and any mixtures thereof.

Surfactants or solubilizers that may be useful in the formulations of the present invention include, but are not limited to: anionic surfactants like potassium laurate, sodium lauryl sulfate (sodium dodecylsulfate), alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, cholic acid and other bile acids (for example, cholic acid, deoxycholic acid, glycocholic acid, taurocholic acid and glycodeoxycholic acid) and salts thereof (for example, sodium deoxycholate); cationic surfactants like quaternary ammonium compounds (e.g., benzalkonium chloride, cetyltrimethylammonium bromide, lauryldimethylbenzylammonium chloride, acyl carnitine hydrochlorides and alkyl pyridinium halides); nonionic surfactants like polyoxyethylene fatty alcohol ethers (MACROGOL™ and BRIJ™), polyoxyethylene sorbitan fatty acid esters (polysorbates or TWEEN™), polyoxyethylene fatty acid esters (MYRJ™), sorbitan esters (SPAN™), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), polaxamines, and the like; and any mixtures thereof.

Pharmaceutical formulations of the present invention may further include any one or more of pharmaceutically acceptable glidants, lubricants like sodium stearyl fumarate, opacifiers, colorants, and other commonly used excipients.

The pharmaceutical formulations of the present invention exhibit desired in vivo absorption profiles for drugs delivered. The in vivo pharmacokinetic parameters frequently used to evaluate pharmaceutical formulations after oral administration include maximum plasma concentration (“Cₘₐₓ”), time after administration until the maximum plasma concentration (“Tₘₐₓ”), area under the plasma concentration-time plot curve (“AUC”), and the like.

The pharmaceutical formulations of the invention may contain one or more active ingredients in addition to niacin. Non-limiting examples of such additional active ingredients include lipid lowering agents, anti-diabetic compounds, NSAIDs, cox-2 inhibitors, PGD2 antagonists to control the flushing, anti-arrhythmic agents, anti-coagulants, anti-depressants, anti-hypertensive agents, α-glucosidase inhibitors, immunosuppressants, anti-thyroid agents, sedatives, hypnotics, beta-blockers, cardiac ionotropic agents, corticosteroids, diuretics, anti-anginal agents, muscle relaxants, nutritional agents, opioid analgesics, muscle relaxants, cognition enhancers, cholesterol absorption inhibitors, bile acid sequestering agents, and the like. Typically, lipid lowering compounds include statins, fibrates and PPAR agonists. Exemplary statins include atorvastatin, simvastatin, lovastatin, pravastatin, cervastatin, fluvastatin, while fibrates comprise fenofibrate, gemfibrozil, and bezafibrate. Non-limiting examples of DPP IV inhibitors include sitagliptan, vildagliptan, saxagliptan. Typical anti-diabetic compounds include sulfonylureas, meglitinides, DPP-IV inhibitors, biguanides, peroxisome proliferator activated receptor (“PPAR”) agonists, glucose uptake modulators. Cholesterol absorption inhibitors include ezetimibe, and the like. Bile acid sequestering agents include orlistat, and the like.

The pharmaceutical formulations disclosed herein can be advantageously used for the treatment of hyperlipidemia, hypercholesterolemia and mixed dyslipidemia, myocardial infarction, atherosclerotic diseases, and other such conditions for the treatment of which niacin finds use.

Clinical studies are conducted to evaluate several characteristic properties of the formulations of present invention, involving dosing a group of healthy volunteers with formulations of the present invention and commercially available formulations such as NIACOR™ (IR tablets from Upsher-Smith Laboratories, Inc.) and NIASPAN™ (IR tablets from Kos Pharmaceuticals Inc.).

Studies are carried out to measure steady state drug levels in plasma:

a. Single dose plasma levels of niacin from NIACOR.

b. Single dose and steady state plasma levels of nicotinuric acid (NUA) and nicotinamide (NAM) from the formulations of the present invention.

c. Single dose and steady state plasma levels of nicotinuric acid (NUA) and nicotinamide from NIASPAN.

d. Single dose and steady state response of cutaneous flushing from the dosing of formulations of the present invention and NIASPAN.

e. Single dose and steady state urinary metabolites from the formulations of the present invention and NIASPAN.

The studies reveal some unique and proprietary features of the formulations of present invention, dosed in lower amounts as compared with NIASPAN:

a. Plasma levels of niacin and nicotinuric acid are significantly high.

b. Significantly low plasma levels of nicotinamide (NAM), a pathway 2-phase I metabolite responsible for production of subsequent metabolites that produce hepatotoxicity, are found.

Accordingly, in an embodiment, the invention provides modified release formulations comprising 500 mg niacin, the formulations providing higher exposure of niacin in plasma, as compared to the exposure obtained after oral administration of 500 mg of niacin from the commercially available NIASPAN intermediate release product.

According to an embodiment, modified release formulations of the present invention, upon administration of 2×500 mg niacin orally, provide higher exposure of niacin in plasma as compared to the exposure obtained after oral administration of 2×500 mg of niacin from the commercially available NIASPAN intermediate release product.

In an embodiment, modified release formulations of the present invention, upon administration of 2×500 mg of niacin provide comparable or higher exposures of niacin in plasma, as compared to the exposure obtained after oral administration of 2×750 mg of niacin from the commercially available NIASPAN intermediate release product.

In an embodiment, modified release formulations of the present invention, upon administration of 2×500 mg of niacin provide higher exposure of nicotinuric acid (NUA) in plasma, as compared to the exposure obtained after oral administration of 2×500 mg of niacin from the commercially available NIASPAN intermediate release product.

In an embodiment, modified release formulations of the present invention, upon administration of 2×500 mg niacin provide comparable or higher exposure of nicotinuric acid (NUA) in plasma, as compared to the exposure obtained after oral administration of 2×750 mg of niacin from the commercially available NIASPAN intermediate release product.

In an embodiment, modified release formulations of the present invention, upon administration of 2×500 mg niacin provide comparable or lower exposure of nicotinamide (NAM) in plasma as compared to the exposure obtained after oral administration of 2×500 mg of niacin from the commercially available NIASPAN intermediate release product.

In an embodiment, modified release formulations of the present invention, upon administration of 2×500 mg niacin provide lower exposure of nicotinamide (NAM) in plasma, as compared to the exposure obtained after oral administration of 2×750 mg of niacin from the commercially available NIASPAN intermediate release product.

In embodiments, the present pharmaceutical formulations comprising niacin in a matrix that modifies release of niacin by incorporating a hydrophobic material provide, following administration to a healthy human, at least one of the pharmacokinetic parameters Cₘₐₓ and AUC_{0-∞} higher than a corresponding value obtained from administering a pharmaceutical formulation containing a similar or greater amount of niacin in a matrix that modifies release of niacin by incorporating a hydrophilic polymer.

In embodiments, the present pharmaceutical formulations comprising niacin in a matrix that modifies release of niacin by incorporating a hydrophobic material provide, following administration to a healthy human, at least one of the pharmacokinetic parameters Cₘₐₓ and AUC_{0-∞} higher than a corresponding value obtained from administering a pharmaceutical formulation containing at least about 25%, 30%, 35%, or 40% more niacin in a matrix that modifies release of niacin by incorporating a hydrophilic polymer.

The following examples are provided to illustrate certain specific aspects and embodiments of the invention and demonstrate the practice and advantages thereof. It is to be understood that the examples are given for purposes of illustration only and are not intended to limit the scope of the invention in any manner.

### Examples 1-5

### Delayed-Extended Release Formulations Comprising Niacin

| | mg/Tablet | | | | |
|---|---|---|---|---|---|
| Ingredient | 1 | 2 | 3 | 4 | 5 |
| Niacin | 500 | 500 | 500 | 500 | 500 |
| Microcrystalline cellulose | 280 | 50 | 50 | — | 50 |
| (Avicel ™ PH101) | | | | | |
| Microcrystalline cellulose | — | — | — | 50 | — |
| (Avicel PH112) | | | | | |
| Anhydrous lactose | — | 50 | 25 | — | — |
| Tablettose ™ 70* | — | — | — | 50 | — |
| Croscarmellose sodium | 25 | 5 | — | 5 | — |
| Eudragit ™ NM 30 D | — | 16 | 16 | 16 | 13.75 |
| Stearic acid | — | 6.5 | 6 | 6.5 | 5.63 |
| Eudragit L 100-55 | 50 | — | — | — | — |
| Triethyl citrate | 12 | — | — | — | — |
| Isopropyl alcohol‡ | 86.5 | — | — | — | — |
| HPMC 6 cps | — | — | — | 12.5 | — |
| Isopropyl alcohol‡ | — | — | — | 25 | — |
| Water‡ | 37.5 | — | — | 10 | — |
| Talc | 8.7 | — | — | — | — |
| Eudragit L 100-55 | — | — | — | 51 | — |
| Isopropyl alcohol‡ | — | — | — | 860 | — |

‡Evaporates during processing.
*Tablettose 70 is an agglomerated α-lactose monohydrate, sold by Meggle Pharma.

### Manufacturing Process for Example 1

1. A blend of niacin, microcrystalline cellulose and croscarmellose sodium is passed through a BSS #60 mesh sieve and mixed in a blender, then is compressed into tablets using 19×8 mm punches.

Coating

2. Eudragit L 100-55 solution is prepared in isopropyl alcohol and water with stirring. Triethyl citrate and talc are added, with stirring.

3. Core tablets of 1 are coated with the solution of 2, to produce an 8% weight increase.

### Manufacturing Process for Examples 2-5

1. Niacin, microcrystalline cellulose and lactose are mixed together and passed through a BSS #60 mesh sieve. The powder mixture is again blended in a blender to attain uniformity.

2. The blend is granulated in a rapid mixer granulator (RMG) using a Eudragit NM 30 D dispersion.

3. Wet granules from 2 are dried and passed through a BSS #24 mesh sieve.

4. Stearic acid is passed through a BSS #60 mesh sieve and mixed with granules from 3.

5. The blended granules of 4 are compressed into tablets using 19×8 mm punches.

Coating

6. A Eudragit L 100-55 solution is prepared in isopropyl alcohol with stirring. Triethyl citrate is added, where required.

7. Core tablets of 5 are coated with the solution of 6, to produce an 8% weight increase.

8. HPMC 6 cps is dissolved in a mixture of isopropyl alcohol and water, and coated onto the tablets from 7, to produce a 2% weight gain.

9. For Example 5, meloxicam and HPMC 6 cps are dissolved in isopropyl alcohol and water, and coated onto tablets from 8.

10. HPMC 6 cps is dissolved in isopropyl alcohol and water, and coated onto tablets from 9, to produce a 2% weight gain.

In vitro release profiles of niacin from the formulations of Examples 1 and 4 are determined using a 0.1 N hydrochloric acid medium for an initial 2 hours, followed by a phosphate buffer pH 6.8 medium for the remainder of the test, using apparatus 2 (paddle) and 75 rpm stirring, with the procedure of Test 711 “Dissolution” in United States Pharmacopeia 29, United States Pharmacopeial Convention, Inc., Rockville, Md., 2005 (“USP”).

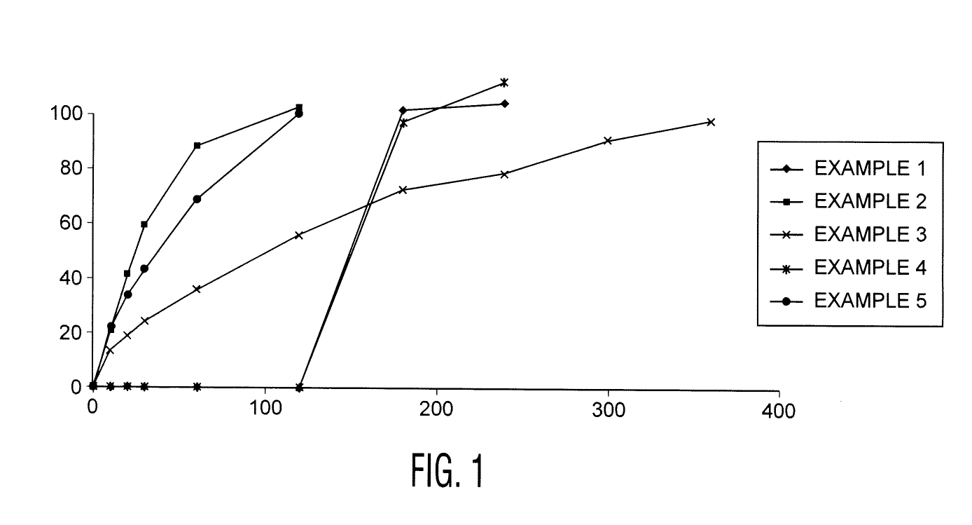

In vitro release profiles of niacin from the formulations of Examples 2, 3, and 5 are determined using the same conditions and procedure described for Examples 1 and 4, except the medium used is 0.001 N HCl (pH 3.0) for the duration of the test. The results are illustrated in FIG. 1, where the vertical axis is cumulative percentage of contained niacin that dissolves, and the horizontal axis is minutes.

### Example 6

### Extended Release Formulation Comprising Niacin 500 mg.

| Ingredient | mg/Tablet |
|---|---|
| Niacin | 500 |
| Microcrystalline cellulose | 50 |
| (Avicel PH112) | |
| Lactose monohydrate | 50 |
| Eudragit NM 30 D* | 32 |
| Croscarmellose sodium | 5 |
| Stearic acid | 6.5 |
| HPMC 6 cps | 19.3 |
| Isopropyl alcohol‡ | 38.6 |
| Water‡ | 15.5 |
| HPMC phthalate | 63.52 |
| Isopropyl alcohol‡ | 256 |
| Water‡ | 102 |
| Triethyl citrate | 9.07 |
| Sodium bicarbonate | 20.17 |
| HPMC 6 cps | 22.66 |
| Isopropyl alcohol‡ | 45.2 |
| Water‡ | 26.6 |

‡Evaporates during processing.
*EUDRAGIT NM 30 D is an aqueous dispersion containing about 30% by weight of a copolymer of ethyl acrylate and methyl methacrylate, having an average molecular weight about 600,000.

Manufacturing Process:

1. Niacin, microcrystalline cellulose and lactose monohydrate are mixed together and passed through a BSS #60 mesh sieve. The powder mixture is again blended in a blender to attain uniformity.

2. The blend of 11s granulated in an RMG using a Eudragit NM 30 D dispersion.

3. The wet granules from 2 are dried and passed through a BSS #24 mesh sieve.

4. Croscarmellose sodium and stearic acid are passed through a BSS #60 mesh sieve and mixed with granules from 3.

5. The blended granules of 4 are compressed into tablets using 19×8 mm punches.

Coating

6. A sub-coating solution is prepared by dispersing HPMC 6 cps (first quantity) in isopropyl alcohol and water, with stirring until a clear solution formed.

7. The coating solution obtained from 6 is coated onto tablets prepared in 5.

8. An enteric coating solution is prepared by dispersing HPMC pthalate, triethyl citrate, and sodium bicarbonate in a mixture of isopropyl alcohol and water, with stirring.

9. Sub-coated tablets from 7 are coated with coating solution of 8.

10. HPMC 6 cps (second quantity) is dissolved in isopropyl alcohol and water, and coated onto the enteric coated tablets of 9 to produce a 2% weight gain.

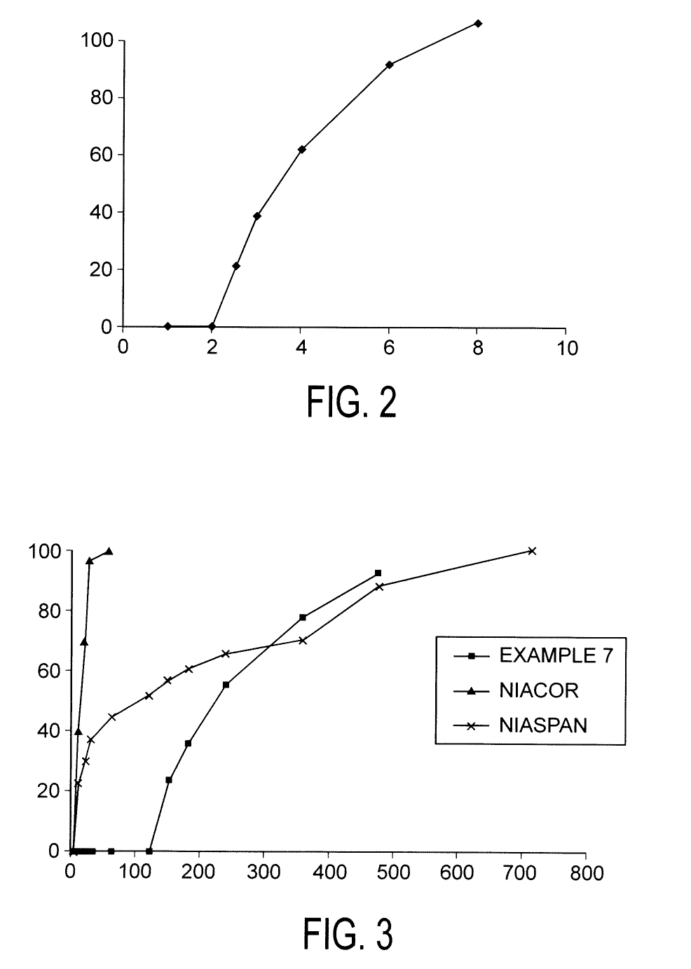

An in vitro release profile of niacin from the formulation of Example 6 is determined using 0.1 N hydrochloric acid for an initial 2 hours, followed by phosphate buffer pH 6.8 for the duration of the test, using apparatus 2 (paddle) and 75 rpm stirring, with the procedure of Test 711 “Dissolution” in United States Pharmacopeia 29, United States Pharmacopeial Convention, Inc., Rockville, Md., 2005 (“USP”). The dissolution profile results are illustrated in FIG. 2, where the vertical axis is cumulative percentage of contained niacin that dissolves, and the horizontal axis is hours.

A two-way crossover pharmacokinetic study is conducted by administering 500 mg tablets of Example 6 and NIACOR™ 500 mg tablets in the morning to 10 healthy human volunteers. After overnight fasting, the test and the reference samples are randomly distributed to the subjects and are swallowed with 250 mL of water. The subjects are asked to maintain a sitting position during the dosing. The results are shown below, where CV is coefficient of variation and n is the number of subjects for the calculation.

| | | | | |
|---|---|---|---|---|
| | AUC₍₀₋ₜ₎ | AUC_{(0-∞)} | Cₘₐₓ | Tₘₐₓ |
| Value | (ng · hr/mL) | (ng · hr/mL) | (ng/mL) | (hours) |

NIACOR 500 mg

| | | | | |
|---|---|---|---|---|
| Mean | 8469.41 | 8522.88 | 6511.62 | 1 |
| % CV | 37.4 | 36.7 | 39.4 | 51.4 |
| n | 10 | 10 | 10 | 10 |

Example 6

| | | | | |
|---|---|---|---|---|
| Mean | 525.34 | 854.39 | 595.61 | 2.5 |
| % CV | 82.8 | 69.7 | 89.5 | 47 |
| n | 10 | 6 | 10 | 10 |

The data indicate a lower maximum exposure of niacin in plasma from the enteric coated formulation, when compared with the NIACOR formulation. A delay in achieving the maximum plasma concentration is seen from the test formulation.

### Examples 7

### Extended Release Formulations Comprising Niacin 500 mg.

| Ingredient | mg/Tablet |
|---|---|
| Niacin | 500 |
| Microcrystalline cellulose | 50 |
| (Avicel PH112) | |
| Lactose monohydrate | 50 |
| Eudragit NM 30 D | 16 |
| Croscarmellose sodium | 5 |
| Stearic acid | 6.5 |
| Opadry ™ Yellow 03B82626* | 18.82 |
| Water‡ | 54.1 |
| HPMC phthalate | 26.55 |
| Isopropyl alcohol‡ | 107 |
| Water‡ | 42.5 |
| Triethyl citrate | 3.79 |
| Sodium bicarbonate | 8.43 |
| Opadry Yellow 03B82626 | 37.68 |
| Water‡ | 120 |

*Opadry Yellow is a product of Colorcon.
‡Evaporates during processing.

Manufacturing Process:

1. Niacin, microcrystalline cellulose and lactose monohydrate are mixed together and passed through a BSS #60 mesh sieve. The powder mixture is again blended in a blender to attain uniformity.

2. The blend of 11s granulated in an RMG using a Eudragit NM 30 D dispersion.

3. The wet granules from 2 were dried and passed through a BSS #24 mesh sieve.

4. Croscarmellose sodium and stearic acid are passed through a BSS #60 mesh sieve and mixed with granules from 3.

5. The blended granules of 4 are compressed into tablets using 19×8 mm punches.

Coating

6. A sub-coating dispersion was prepared by dispersing Opadry Yellow (first quantity) in water, with stirring until a uniform dispersion is obtained.

7. The opadry dispersion obtained from 6 is coated onto tablets prepared in 5.

8. An enteric coating solution is prepared by dispersing HPMC pthalate, triethyl citrate, and sodium bicarbonate in a mixture of isopropyl alcohol and water, with stirring.

9. Sub-coated tablets from 7 are coated with coating solution of 8.

10. Opadry Yellow (second quantity) was dispersed in water and stirred until a uniform dispersion is obtained. This dispersion was coated onto the enteric coated tablets of 9 to produce a 2% weight gain.

The in vitro release profiles of niacin for the Example 7 product is determined using a 0.1 N hydrochloric acid medium for an initial 2 hours, followed by a phosphate buffer pH 6.8 medium for the remainder of the test, using apparatus 2 (paddle) and 75 rpm stirring, with the procedure of Test 711 “Dissolution” in United States Pharmacopeia 29, United States Pharmacopeial Convention, Inc., Rockville, Md., 2005 (“USP”). Samples of NIACOR 500 mg and NIASPAN 500 mg tablets are also tested, for comparison.

The dissolution profile results are illustrated in FIG. 3, where the vertical axis is cumulative percentage of contained niacin that dissolves, and the horizontal axis is minutes.

### Example 8

### Pharmacokinetic Study

A three-way crossover study is conducted by administering 2×500 mg tablets of Example 6, Example 7, and NIASPAN in the morning, to 23 healthy human volunteer subjects after overnight fasting. The test and the reference samples are randomly distributed to the subjects and are swallowed with 250 mL of water. The subjects are asked to maintain a sitting posture during the dosing. Results are as shown below.

| | | | | |
|---|---|---|---|---|
| | AUC₍₀₋ₜ₎ | AUC_{(0-∞)} | Cₘₐₓ | Tₘₐₓ |
| Value | (ng · hr/mL) | (ng · hr/mL) | (ng/mL) | (hours) |

NIASPAN

| | | | | |
|---|---|---|---|---|
| Mean | 445.35 | 754.14 | 506.55 | 1.75 |
| % CV | 84.1 | 65.1 | 86.7 | 91.3 |
| n | 17 | 8 | 19 | 19 |

Example 6

| | | | | |
|---|---|---|---|---|
| Mean | 7924.81 | 10957.92 | 5114.86 | 3 |
| % CV | 153 | 125 | 123.4 | 73.5 |
| n | 19 | 13 | 19 | 19 |

Example 7

| | | | | |
|---|---|---|---|---|
| Mean | 22137.34 | 28187.35 | 12482.71 | 3 |
| % CV | 88.6 | 63.2 | 80.6 | 61.8 |
| n | 19 | 15 | 19 | 19 |

The data indicate that significantly higher exposures, in terms of elevated niacin Cₘₐₓ and AUC values, are obtained with the formulations of the examples when compared with NIASPAN.

### Example 9

### Pharmacokinetic Study

A three-way crossover study is conducted using 2×500 mg tablets of Example 6, Example 7, and NIASPAN in the morning, with 24 healthy human volunteer subjects. The subjects fast overnight, then are administered a standard meal 30 minutes before dosing. The test and reference samples are randomly distributed to the subjects and are swallowed with 250 mL of water. The subjects are asked to maintain a sitting position during the dosing.

| | | | | |
|---|---|---|---|---|
| | AUC₍₀₋ₜ₎ | AUC_{(0-∞)} | Cₘₐₓ | Tₘₐₓ |
| Value | (ng · hr/mL) | (ng · hr/mL) | (ng/mL) | (hours) |

NIASPAN

| | | | | |
|---|---|---|---|---|
| Mean | 4966.22 | 2885.42 | 3913.14 | 5.13 |
| % CV | 102.48 | 67.78 | 110.99 | 47.91 |
| n | 11 | 4 | 11 | 11 |

Example 6

| | | | | |
|---|---|---|---|---|
| Mean | 18725.43 | 25283.23 | 10712.45 | 5.09 |
| % CV | 87.65 | 84.71 | 67.65 | 38.75 |
| n | 11 | 5 | 11 | 11 |

Example 7

| | | | | |
|---|---|---|---|---|
| Mean | 28962.15 | 36210.89 | 16379.85 | 4.04 |
| % CV | 63.43 | 46.15 | 59.81 | 44.03 |
| n | 11 | 8 | 11 | 11 |

The data indicate that significantly higher exposures, in terms of elevated Cₘₐₓ and AUC values, are obtained with the formulations of the examples, as compared to NIASPAN.

### Example 10

### Extended Release Formulation Comprising Niacin 500 mg.

| Ingredient | mg/Tablet |
|---|---|
| Niacin | 500 |
| Microcrystalline cellulose | 50 |
| (Avicel PH-112) | |
| Lactose monohydrate | 50 |
| Eudragit NM 30 D | 32 |
| Croscarmellose sodium | 5 |
| Stearic acid | 6.5 |
| HPMC 6 cps | 19.3 |
| Isopropyl alcohol‡ | 38.6 |
| Water‡ | 15.5 |
| HPMC phthalate | 71.39 |
| Isopropyl alcohol‡ | 256 |
| Water‡ | 102 |
| Triethyl citrate | 7.13 |
| Sodium bicarbonate | 14.27 |
| Opadry Orange 03K93365* | 22.66 |
| Water‡ | 26.6 |

*Opadry Orange is a product of Colorcon.
‡Evaporates during processing.

Manufacturing Process:

Steps 1-5 are similar to those of Example 7.

Coating

6. A sub-coating solution was prepared by dissolving HPMC 6 cps in isopropyl alcohol and water, with stirring until a clear solution is obtained.

7. The coating solution obtained from 6 is coated onto tablets prepared in 5.

8. An enteric coating solution is prepared by dispersing HPMC pthalate, triethyl citrate, and sodium bicarbonate in a mixture of isopropyl alcohol and water, with stirring.

9. Sub-coated tablets from 7 are coated with coating solution of 8.

10. Opadry Orange was dispersed in water and stirred until a uniform dispersion is obtained. This dispersion was coated onto the enteric coated tablets of 9 to produce a 2% weight gain.

A randomised, double-blind, crossover study is conducted at a single study centre using Example 10 (2×500 mg of niacin) and NIASPAN (2×750 mg of niacin) products, administered once-daily for 3 days in each period. Clinical assessment for safety is performed during a stay in the clinic for all three periods. Pharmacokinetic assessment is done using samples collected at the following time points after dose administration on Day 1 and Day 3: 0.5, 1, 2, 3, 4, 5, 6, 8, 10, and 12 hours for plasma; and 0-6, 6-12, 12-18, and 18-24 hours for urine.

The study related to pharmacokinetic assessment results in a large variation of parameters in terms of Cₘₐₓ, Tₘₐₓ and AUC. The Cₘₐₓ values for niacin and its metabolites for Day 1 and Day 3 are comparable for Example 10, and NIASPAN. The exposures to NUA and NAM are higher on Day 3 compared to Day 1 for both Example 10 and NIASPAN. The exposures to niacin and NUA are higher for Example 10 compared to NIASPAN, on Day 1 and Day 3. The exposures to NAM are higher for NIASPAN compared to Example 10, on Day 1 and Day 3.

From the results it can be concluded that:

a. The Cₘₐₓ and exposure to niacin is higher for Example 10, compared to NIASPAN.

b. The exposure to NAM is lower for Example 10, compared to NIASPAN.

c. The exposure to NUA is lower in NIASPAN, compared to Example 10.

d. The Tₘₐₓ for niacin is about 4.8 hours for Example 10 and NIASPAN.

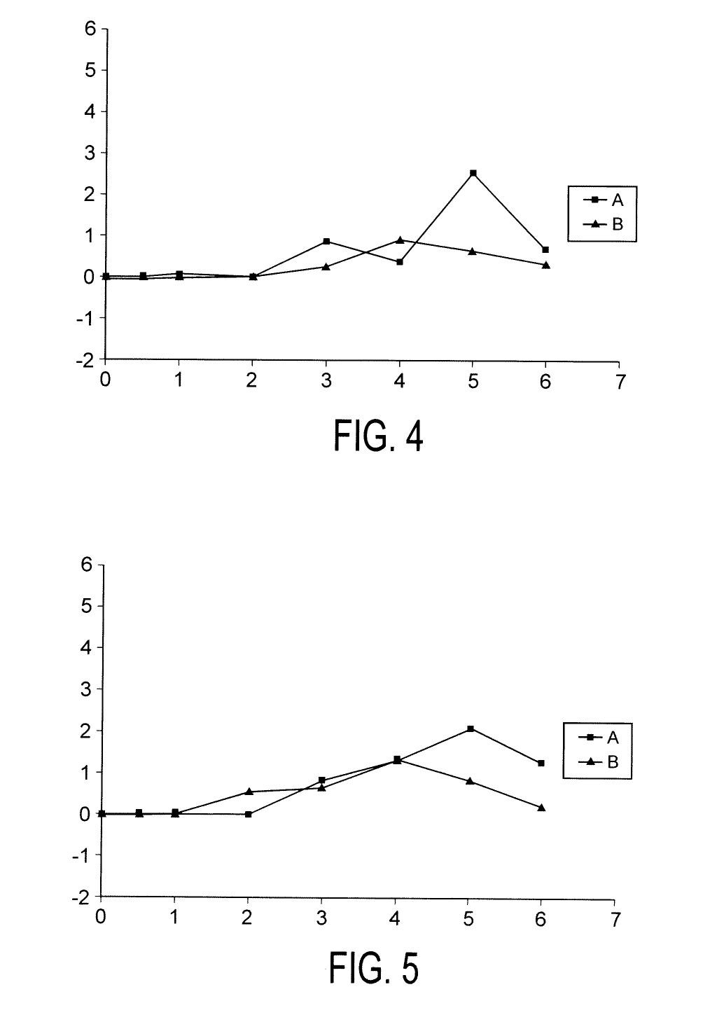

The mean plasma concentrations of niacin and its metabolites in terms of Cₘₐₓ, AUC and Tₘₐₓ from the study are shown in Tables 1-3 and are illustrated in FIGS. 4-9.

In FIG. 4, the vertical axis is plasma concentration of niacin on day 1, in μg/mL, and the horizontal axis is hours. The data points identified as “A” are for Example 10 and the data points identified as “B” are for NIASPAN.

In FIG. 5, the vertical axis is plasma concentration of niacin on day 3, in μg/mL, and the horizontal axis is hours. The data points identified as “A” are for Example 10 and the data points identified as “B” are for NIASPAN.

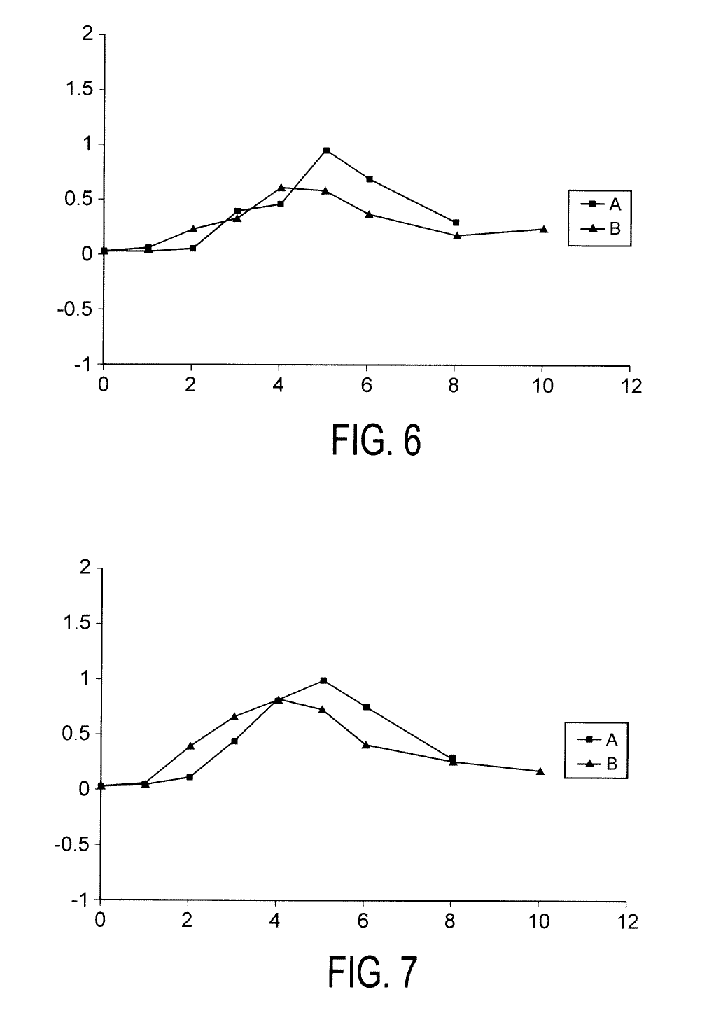

In FIG. 6, the vertical axis is plasma concentration of NUA on day 1, in μg/mL, and the horizontal axis is hours. The data points identified as “A” are for Example 10 and the data points identified as “B” are for NIASPAN.

In FIG. 7, the vertical axis is plasma concentration of NUA on day 3, in μg/mL, and the horizontal axis is hours. The data points identified as “A” are for Example 10 and the data points I identified as “B” are for NIASPAN.

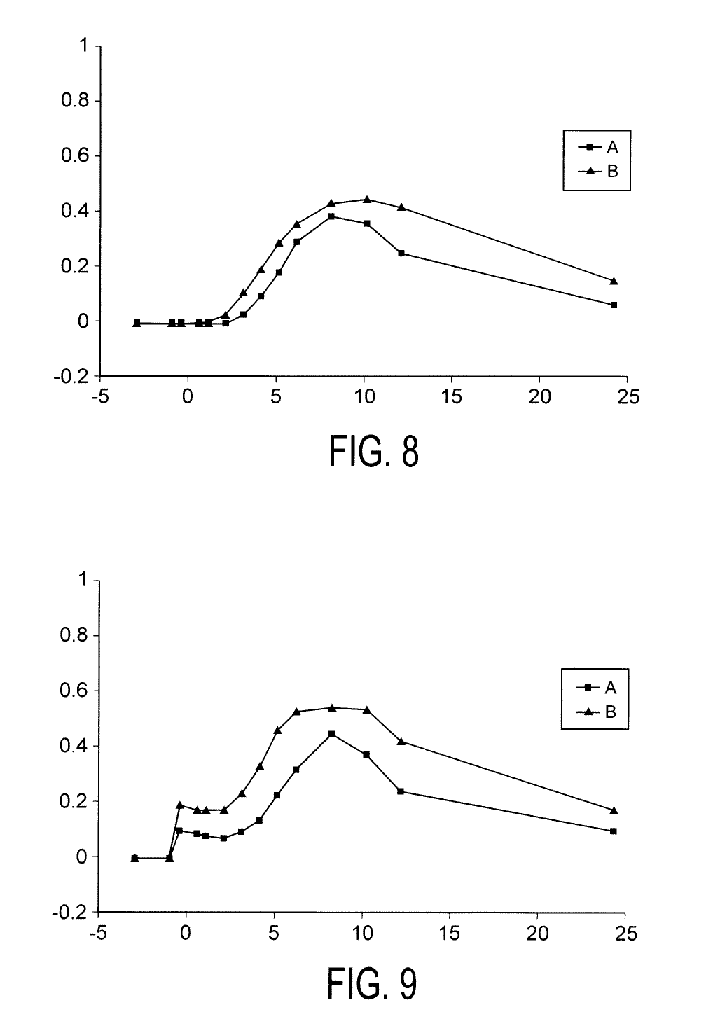

In FIG. 8, the vertical axis is plasma concentration of NAM on day 1, in μg/mL, and the horizontal axis is hours. The data points identified as “A” are for Example 10 and the data points identified as “B” are for NIASPAN.

In FIG. 9, the vertical axis is plasma concentration of NAM on day 3, in μg/mL, and the horizontal axis is hours. The data points identified as “A” are for Example 10 and the data points identified as “B” are for NIASPAN.

TABLE 1
Pharmacokinetic parameters for niacin.

| | | | | |
|---|---|---|---|---|
| | AUC₍₀₋ₜ₎ | AUC_{(0-∞)} | Cₘₐₓ | Tₘₐₓ |
| Value | (ng · hr/mL) | (ng · hr/mL) | (ng/mL) | (hours) |

Example 10 on Day 1

| | | | | |
|---|---|---|---|---|
| Mean | 4352 | — | 3506 | 4.83 |
| % CV | — | — | — | — |
| n | 18 | 18 | 18 | 18 |

NIASPAN on Day 1

| | | | | |
|---|---|---|---|---|
| Mean | 3300 | — | 1899 | 4.88 |
| % CV | 297.7 | — | 238.3 | — |
| n | 17 | — | 17 | 17 |

Example 10 on Day 3

| | | | | |
|---|---|---|---|---|
| Mean | 4862 | — | 3139 | 4.87 |
| % CV | — | — | — | — |
| n | 17 | — | 17 | 17 |

NIASPAN on Day 3

| | | | | |
|---|---|---|---|---|
| Mean | 4241 | — | 2379 | 4.94 |
| % CV | 148.9 | — | 147.7 | — |
| n | 17 | — | 17 | 17 |

TABLE 2
Pharmacokinetic parameters for nicotinuric acid (NUA).

| | | | | |
|---|---|---|---|---|
| | AUC₍₀₋ₜ₎ | AUC_{(0-∞)} | Cₘₐₓ | Tₘₐₓ |
| Value | (ng · hr/mL) | (ng · hr/mL) | (ng/mL) | (hours) |

Example 10 on Day 1

| | | | | |
|---|---|---|---|---|
| Mean | 3266 | 4901 | 1261 | 4.95 |
| % CV | 55.2 | 36.7 | 57.1 | — |
| n | 18 | 8 | 18 | 18 |

NIASPAN on Day 1

| | | | | |
|---|---|---|---|---|
| Mean | 2921 | 2573 | 920.5 | 4.76 |
| % CV | 78.3 | 78.4 | 60.8 | — |
| n | 17 | 9 | 17 | 17 |

Example 10 on Day 3

| | | | | |
|---|---|---|---|---|
| Mean | 3550 | 5599 | 1190 | 5.29 |
| % CV | 54.9 | 32.7 | 55.4 | — |
| n | 17 | 7 | 17 | 17 |

NIASPAN on Day 3

| | | | | |
|---|---|---|---|---|
| Mean | 3582 | 3999 | 1030 | 4.76 |
| % CV | 48.7 | 47.2 | 43.3 | — |
| n | 17 | 9 | 17 | 17 |

TABLE 3
Pharmacokinetic parameters for nicotinamide (NAM).

| | | | | |
|---|---|---|---|---|
| | AUC₍₀₋ₜ₎ | AUC_{(0-∞)} | Cₘₐₓ | Tₘₐₓ |
| Value | (ng · hr/mL) | (ng · hr/mL) | (ng/mL) | (hours) |

Example 10 on Day 1

| | | | | |
|---|---|---|---|---|
| Mean | 4226 | 6278 | 460.4 | 8.22 |
| % CV | 66.7 | 62 | 66.4 | — |
| n | 18 | 9 | 18 | 18 |

NIASPAN on Day 1

| | | | | |
|---|---|---|---|---|
| Mean | 6896 | 11820 | 581 | 8.17 |
| % CV | 75 | 109.8 | 86.5 | — |
| n | 17 | 2 | 17 | 17 |

Example 10 on Day 3

| | | | | |
|---|---|---|---|---|
| Mean | 5174 | 7601 | 497.8 | 7.94 |
| % CV | 44.7 | 35.4 | 51.1 | — |
| n | 17 | 7 | 17 | 17 |

NIASPAN on Day 3

| | | | | |
|---|---|---|---|---|
| Mean | 8540 | 13650 | 682.1 | 8.17 |
| % CV | 63.7 | 36 | 65.9 | — |
| n | 17 | 3 | 17 | 17 |

1. A pharmaceutical formulation comprising niacin in a matrix that modifies release of niacin by incorporating a hydrophobic material, the formulation providing, following administration to a healthy human, at least one of the pharmacokinetic parameters Cₘₐₓ and AUC_{0-∞} greater than a corresponding value obtained from administering a pharmaceutical formulation containing a similar or greater amount of niacin in a matrix that modifies release of niacin by incorporating a hydrophilic polymer.

2. The pharmaceutical formulation of claim 1, wherein at least one of the pharmacokinetic parameters Cₘₐₓ and AUC_{0-∞} is greater than a corresponding value obtained from administering a pharmaceutical formulation containing at least about 25 percent more niacin in a matrix that modifies release of niacin by incorporating a hydrophilic polymer.

3. The pharmaceutical formulation of claim 1, wherein at least one of the pharmacokinetic parameters Cₘₐₓ and AUC_{0-∞} is greater than a corresponding value obtained from administering a pharmaceutical formulation containing at least about 35 percent more niacin in a matrix that modifies release of niacin by incorporating a hydrophilic polymer.

4. The pharmaceutical formulation of claim 1, wherein a hydrophobic material comprises a hydrophobic polymer.

5. The pharmaceutical formulation of claim 1, wherein a hydrophobic material comprises a hydrophobic polymer comprising a copolymer of an alkyl acrylate and an alkyl methacrylate, alkyl groups being the same or different and having 1 to about 4 carbon atoms.

6. The pharmaceutical formulation of claim 1, wherein a hydrophobic material comprises a hydrophobic polymer comprising a 1:1 copolymer of ethyl acrylate and methyl methacrylate, having an average molecular weight about 600,000.

7. The pharmaceutical formulation of claim 1, wherein a matrix is formed by a method comprising granulating a solid mixture comprising niacin with a liquid comprising a hydrophobic polymer.

8. The pharmaceutical formulation of claim 1, wherein a matrix is formed by a method comprising granulating a particulate mixture comprising niacin with an aqueous suspension comprising a hydrophobic polymer.

9. The pharmaceutical formulation of claim 1, wherein a matrix is formed by a method comprising combining a particulate mixture comprising niacin with a hydrophobic polymer.

10. The pharmaceutical formulation of claim 1, wherein a hydrophobic material comprises a fatty substance or a mineral.

11. The pharmaceutical formulation of claim 1, having a first polymer coating disposed over a tablet formed using a matrix comprising a hydrophobic polymer.

12. The pharmaceutical formulation of claim 1, having a first polymer coating comprising a cellulose derivative, disposed over a tablet formed using a matrix comprising a hydrophobic polymer.

13. The pharmaceutical formulation of claim 1, having a first polymer coating comprising a hydroxypropyl methylcellulose, disposed over a tablet formed using a matrix comprising a hydrophobic polymer.

14. The pharmaceutical formulation of claim 11, having a coating of an enteric polymer disposed over a first polymer coating.

15. The pharmaceutical formulation of claim 11, having a coating of a copolymer of methacrylic acid, or an alkyl methacrylate, and an alkyl acrylate, disposed over a first polymer coating.

16. The pharmaceutical formulation of claim 11, having a coating of a hydroxypropyl methylcellulose phthalate disposed over a first polymer coating.

17. A pharmaceutical formulation comprising niacin in a matrix that modifies release of niacin by incorporating a hydrophobic material, and a first polymer coating disposed over a tablet formed using the matrix, the formulation providing, following administration to a healthy human, at least one of the pharmacokinetic parameters Cₘₐₓ and AUC_{0-∞} greater than a corresponding value obtained from administering a pharmaceutical formulation containing a similar or greater amount of niacin in a matrix that modifies release of niacin by incorporating a hydrophilic polymer.

18. The pharmaceutical formulation of claim 17, wherein at least one of the pharmacokinetic parameters Cₘₐₓ and AUC_{0-∞} is greater than a corresponding value obtained from administering a pharmaceutical formulation containing at least about 25 percent more niacin in a matrix that modifies release of niacin by incorporating a hydrophilic polymer.

19. The pharmaceutical formulation of claim 17, wherein a hydrophobic material comprises a copolymer of an alkyl acrylate and an alkyl methacrylate, alkyl groups being the same or different and having 1 to about 4 carbon atoms.

20. The pharmaceutical formulation of claim 17, wherein a hydrophobic material comprises a 1:1 copolymer of ethyl acrylate and methyl methacrylate, having an average molecular weight about 600,000.

21. The pharmaceutical formulation of claim 17, wherein a first polymer coating comprises a cellulose derivative.

22. The pharmaceutical formulation of claim 17, wherein a first polymer coating comprises a hydroxypropyl methylcellulose.

23. The pharmaceutical formulation of claim 17, having a coating of an enteric polymer disposed over a first polymer coating.
